(19) United States
(12) Patent Application Publication
(10) Pub. No.: US 2024/0237943 A1
(43) Pub. Date: Jul. 18, 2024

(54) DEVICE FOR MEASURING PHYSIOLOGICAL PARAMETERS

(71) Applicant: Cosinuss GmbH, München (DE)

(72) Inventor: Johannes KREUZER, München (DE)

(73) Assignee: Cosinuss GmbH, München (DE)

(21) Appl. No.: 18/573,996

(22) PCT Filed: Jun. 23, 2022
(86) PCT No.: PCT/EP2022/067163

§ 371 Date: Dec. 22, 2023

(30) Foreign Application Priority Data
Jun. 23, 2021 (DE) 10 2021 206 461.2

(51) Int. Cl. A61B 5/00; A61B 5/0205

(52) U.S. Cl. CPC A61B 5/6817; A61B 5/02055; A61B 5/6835; A61B 5/0084

(57) ABSTRACT

The invention relates to a device for measuring physiological parameters insertable into an external auditory canal of a human and to a method for measuring physiological parameters in an external auditory canal of a human.

The present disclosure relates to a device for measuring physiological parameters which is insertable into an external auditory canal of a human and to a method for measuring physiological parameters in an external auditory canal of a human.

### STATE OF THE ART

In the prior art, there are already devices known which can be used to measure physiological parameters in an external auditory canal of a human.

As an example of a device of the prior art, the device described in the international publication WO 2020/212483 A1 which can be used to measure physiological parameters, is mentioned.

A problem with the known devices, however, is that the sensor elements must remain in contact with the skin of the external auditory canal so that the physiological parameters, such as pulse frequency, arterial oxygen saturation, breathing frequency, etc., can be measured with a sufficiently high resolution. The problem here is that the ear canals in humans are of different sizes and shapes, so that an individual adaptation of the device to the anatomical conditions of the wearer is necessary.

Another problem with the known devices is that in medical applications for example cross-contamination must be prevented, if a device is to be used by several different patients. However, when using the devices, hygiene standards must be met, which can be made even stricter by the legislature especially in times of pandemics.

### Presentation of the Disclosure, Task, Solution, Advantages

Based on the aforementioned considerations, the present disclosure is therefore based on the object of providing a device and a method which overcomes the above-mentioned disadvantages of the prior art, i.e. in particular enabling a measurement of the physiological parameters, such as pulse frequency, arterial oxygen saturation and respiratory frequency, with high resolution and preventing cross-contamination when used by several different patients.

In a first aspect, the present disclosure therefore relates to a device for measuring physiological parameters. The device can be at least partially insertable into an external auditory canal of a human. The device can have a hose line for insertion into the external auditory canal of a human and a housing connected to the hose line. At least one sensor device for measuring a physiological parameter can be integrated into the housing. The hose line can have a section at a distal end that faces the eardrum when worn. The hose line can be at least partially inserted into the external auditory canal with the section at the distal end. The section can have at least one wing element with at least a first sensor component for measuring physiological parameters. The wing element can have a restoring force, so that, when the device is worn, the at least one first sensor component can be brought into contact with skin of the external auditory canal by the restoring force of the at least one wing element.

In connection with the device according to the disclosure, the term “distal” refers to the distance in the direction of the human eardrum relative to the at least one sensor device integrated into the housing. In the present case, the first sensor component can be understood as any component with which physiological, medical or other data can be measured and converted into electrical signals. Preferably, the first sensor component is a sensor component that requires contact or optical proximity to the skin of the external auditory canal. Examples of the first sensor component are a light sensor and/or a photodiode, preferably combined with a spatially separate LED for the PPG sensor system in order to monitor physiological parameters (e.g. oxygen saturation, pulse rate) to be measured accurately. Preferably, the first sensor component is positioned sitting on part of the wing.

The restoring force of the at least one wing element ensures that the device automatically and comfortably adapts to the user's different ear canals. An individual manual adjustment of the device to the anatomical characteristics of the user's external auditory canal is not necessary.

According to a preferred implementation, the wing element comprises at least one second sensor component, so that, when the device is worn, the at least one second sensor component can be brought into contact with skin of the external auditory canal by the restoring force of the wing element. According to the disclosure, a sensor component as described for the first sensor component can be used as the second sensor component. The first and second sensor components can preferably be positioned at different locations on the wing element and can thereby measure different physiological data.

If the wing element comprises a second sensor component in addition to the at least one first sensor component, physiological data that requires contact or optical proximity to the skin of the external auditory canal can be measured more precisely.

Preferably, the device additionally comprises at least one third sensor component at the distal end of the section. When wearing the device, the at least one third sensor component can be stably positioned within the external auditory canal.

Any component with which physiological, medical or other data can be measured and converted into electrical signals can be used as the third sensor component. Preferably, the third sensor component is a sensor component that does not necessarily require contact or optical proximity to the skin of the external auditory canal and can also be positioned in a central position of the external auditory canal. Examples of the third sensor component are an infrared (IR) temperature sensor; a sound wave sensor or transmitter, for example a loudspeaker or a microphone, a distance sensor, an LED in combination with a photodiode for artifact suppression and ambient light. Preferably, the third sensor component is positioned sitting at the distal end of the section. Preferably, the third sensor component is an IR temperature sensor and/or a sound wave sensor or transmitter.

If the device comprises a third sensor component for a stably positioning in the external auditory canal, physiological data can be measured by multiple sensors without disruption and interference. For example, a signal can be measured by the third sensor component, which enables artifact suppression in the measurement by the at least one first and/or the at least one second sensor component. Advantageously, additional parameters can be measured and evaluated, including bio-feedback or voice for transmission to the user.

Preferably, the device additionally comprises further sensor components at the distal end of the section. Examples of the further sensor components, which can also preferably be arranged hidden within the section behind the third sensor component, are an accelerometer and a gyroscope. If the device comprises additional sensor components, the device can measure additional physiological parameters, such as acceleration forces, changes in the spatial orientation of the device and rotational movements and movements of the user, etc.

According to a preferred implementation, the device additionally comprises an elastic umbrella for positioning in the external auditory canal. The elastic umbrella can be detachably connected to the section.

According to the disclosure, an elastic umbrella is understood to mean any umbrella which, due to its size and shape as well as its elasticity, is suitable for being inserted into the external auditory canal of a person and thereby positioning the device at least partially in a stable manner in the external auditory canal. Preferably, the umbrella has a size and a shape in order to fully or partially accommodate the at least one wing element with the at least one first sensor component therein. Preferably, the elasticity of the umbrella is such that the restoring force of the at least one wing element can make contact between the at least one sensor component and an inner wall of the external auditory canal by elastically deforming the umbrella.

Preferably, the elastic umbrella essentially has the shape of a truncated cone shell, preferably an oval truncated cone shell. Preferably, the top surface of the truncated cone shell has an opening through which physiological data can be measured in the user's external auditory canal using the third sensor component. For example, acoustic waves, such as sound, or light waves can be measured through the opening of the elastic umbrella. If the elastic umbrella essentially has the shape of a truncated cone, the device can be more easily inserted at least partially into the user's external auditory canal and can be stabilized in the external auditory canal by the restoring force of the wing element on the umbrella. The elastic umbrella is preferably constructed essentially from a translucent material. In an alternative implementation, the elastic umbrella can also be constructed to be matt interspersed with a few color pigments.

Preferably, the elastic umbrella has a recess which can be adjusted in size and shape to at least partially accommodate the section therein. As a result, the elastic umbrella can be connected to the section in a detachable manner. In the present disclosure, a recess is understood to mean any recess which, due to its size and dimension, is suitable for at least partially accommodating the section of the device and thereby providing a detachable connection between the umbrella and the section. The detachable connection can preferably be a plug-in connection, i.e. the umbrella is detachably connected to the section by simply plugging it on. Alternatively, the umbrella can also be connected to the section via a screw connection. In other words, the section and the recess are each matched to one another in terms of size and shape, so that a detachable connection of these two parts is made possible by a plug or screw connection. One advantage of the plug-in connection is that it is particularly easy to connect the elastic umbrella to the section. A screw connection, on the other hand, surprisingly creates a particularly strong and secure connection between the elastic umbrella and the section.

Due to the detachable connection between the umbrella and the section, it is possible that the umbrellas are easier to replace and, in medical applications, surprisingly prevent cross-contamination even when a device is to be used by several patients.

The elastic umbrella can preferably have one or more holes (or side openings) so that, when the device is worn, the at least one first and optionally the at least one second sensor component can be brought into contact with an inner wall of the external auditory canal through the holes.

In an alternative implementation, the elastic umbrella can preferably have one or more depressions with a smaller material thickness, so that, when the device is worn, the at least one first and optionally at least one second sensor component can be brought into measuring connection with an inner wall of the external auditory canal via the one or more depressions. In the present disclosure, any sub-element of the elastic umbrella in which material is missing or has been removed and a residual material with a low material thickness remains, can be understood to mean depression. The depression can be created by appropriately removing material up to the desired thickness of the residual material or when producing the elastic umbrella using the casting process, the depression can also be created by corresponding increases in a negative mold, whereby in the area of depressions a lower material thickness of the residual material can occur in the positive mold after casting.

The elastic umbrella and the recess are preferably constructed in one piece, which has manufacturing advantages. If the elastic umbrella has one or more depressions, then the residual material of the depression(s) is also made of the same material, i.e. all parts of the umbrella are in one piece and made of the same material.

In a preferred implementation, the residual material of the one or more depressions is translucent. The at least one first and optionally the at least one second sensor component can be at least one light sensor. This has the advantage that the sensor components do not have direct contact with the skin of the external auditory canal, but are only positioned near the contact surface of the external auditory canal, i.e. do not touch it hygienically. The elastic umbrella is preferably made of a matt material interspersed with color pigments and is in the depressions manufactured in such a thin material thickness of the residual material that it is highly translucent at this point. Preferably, the umbrella is more translucent in the parts that are not designed as depressions than in the area of the residual material of the depressions. The residual material of the depressions of the umbrella preferably has a material thickness of 10 μm to 2 mm, in particular 100 μm to 500 μm, ideally 200 μm. If the material thickness of the residual material of the depressions lies within this range, there is an optimal compromise between the ease of production of the elastic umbrella on the one hand and the high light transmission of the residual material in the area of the depressions on the other hand.

The device can preferably have exactly one wing element. If the device has exactly one wing element, the device can be manufactured particularly easily in terms of production technology. Alternatively, the device can preferably have at least two wing elements or at least three wing elements. If the device has at least two wing elements or at least three wing elements, then the restoring force of the wing elements can provide more uniform contact with the external auditory canal and hold the device more stably in the user's external auditory canal.

The at least two wing elements can be arranged centrosymmetrically or mirror-symmetrically around a substantially cylindrical section at the distal end. In the case of a centrosymmetric or mirror-symmetrical arrangement of the wing elements, the device is held more stable in the external auditory canal. In the case of at least three wing elements, these can preferably be arranged centrosymmetrically (3-tooth rotational symmetry) around a substantially cylindrical section at the distal end.

In an alternative implementation, the at least two wing elements can also be arranged one behind the other on a substantially cylindrical section be arranged distal end. This results in greater stability because the wing elements are pressed against the inner wall of the auditory ear on only one side with an equal but increased restoring force.

The at least one wing element preferably consists essentially of an elastic material, which can provide a restoring force due to its material properties. For example, the wing element can consist of a dimensionally stable but elastically deformable polymer whose glass transition point is below room temperature. These polymers can deform under tensile and compressive loads, but then return to their original, undeformed shape due to the restoring force. This implementation is preferred because the provision of the wing element with restoring force can be done more easily in terms of manufacturing technology and the device is less susceptible to a failure.

In an alternative implementation, the at least one wing element consists essentially of an inelastic material, for example a hard polymer, and has at least one spring element, for example a spiral spring, to provide a restoring force.

In a second aspect, the present disclosure relates to a method for measuring physiological parameters in an external auditory canal of a human, comprising the following consecutive steps. In a first step (step a)), a device for measuring physiological parameters that can be inserted into an external auditory canal of a human can be provided. In a second step (step b)), the at least one wing element of the section can be moved against its restoring force. If necessary, the section can be detachably connected to the elastic umbrella. In a third step (step c)), the device can be introduced into the external auditory canal of a human being by bringing the at least one first sensor component into contact with an inner wall of the external auditory canal by the restoring force of the wing element. In a fourth step (step d)), physiological parameters in the external auditory canal of the human can be measured by means of the at least one first sensor component.

As used herein, the singular form of the articles “a”, “an” and “the” includes the corresponding plural forms unless otherwise specified. For example, the term “an ear contact surface” encompasses a corresponding ear contact surface or multiple ear contact surfaces in the external auditory canal. As used herein, the term “introduced” or “used” comprises the corresponding partial introduction or insertion unless otherwise stated. For example, the term “introduction into the external auditory canal” of the hose line or the device also encompasses a corresponding partial insertion of the hose line or the device.

The term “insertable into the external auditory canal of a person” of the hose line or the device also encompasses a corresponding partial usability of the hose line or the device.

### BRIEF DESCRIPTION OF THE FIGURES

The disclosure is explained in more detail below using the accompanying schematic drawings. The drawings are not to scale; in particular, for reasons of clarity, the ratios of the individual dimensions to one another do not necessarily correspond to the dimensional ratios in actual technical implementations.

Several preferred exemplary implementations are described, to which the disclosure is, however, not limited. In principle, any variant of the disclosure described or indicated within the scope of the present application can be particularly advantageous, depending on the economic, technical and possibly medical conditions in the individual case. Unless stated otherwise, or as far as technically feasible in principle, individual features of the described implementations are interchangeable or can be combined with each other and with features known per se from the prior art.

In the particular implementations,

FIG. 1 is a side view of an exemplary implementation of the device according to the disclosure for measuring physiological parameters with a wing element.

FIG. 2 is a side view of an exemplary implementation of a device for measuring physiological parameters with two wing elements.

FIG. 3 are detailed views of different implementations of the elastic umbrellas of the device.

FIG. 4 is a detailed view of the elastic umbrella of an implementation of the device with a depression and residual material.

FIG. 5 is a side view of an exemplary implementation of the device according to the disclosure for measuring physiological parameters with an elastic wing element

FIG. 8 is a detailed view of the elastic umbrella of an exemplary implementation of the device with a recess.

FIG. 7 is a partial view of an exemplary implementation of a device for measuring physiological parameters with two wing elements arranged one behind the other and five sensor components.

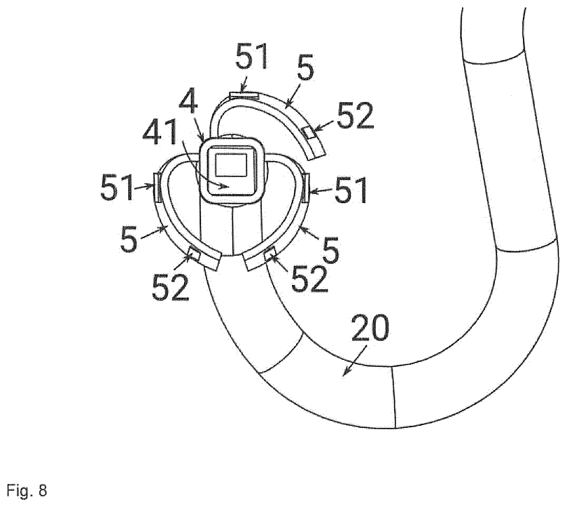

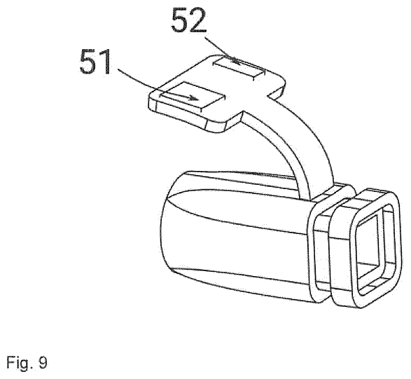

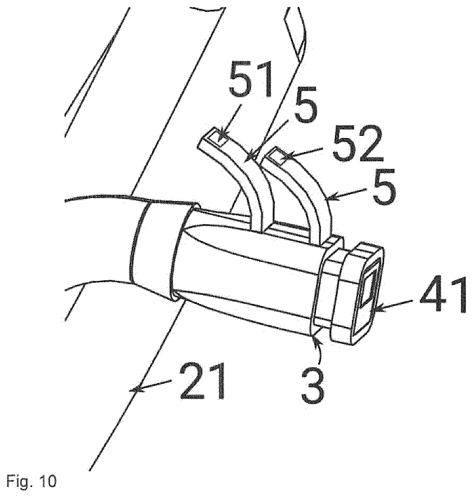

FIG. 8 is a partial view of an exemplary implementation of a device for measuring physiological parameters with three wing elements arranged next to one another and seven sensor components.

FIG. 9 shows a partial view of an exemplary implementation of a device for measuring physiological parameters with a wing element and two different sensor components arranged next to one another.

FIG. 10 shows a partial view of an exemplary implementation of a device for measuring physiological parameters with two wing elements arranged one behind the other and two similar sensor components,

### PREFERRED IMPLEMENTATION OF THE DISCLOSURE

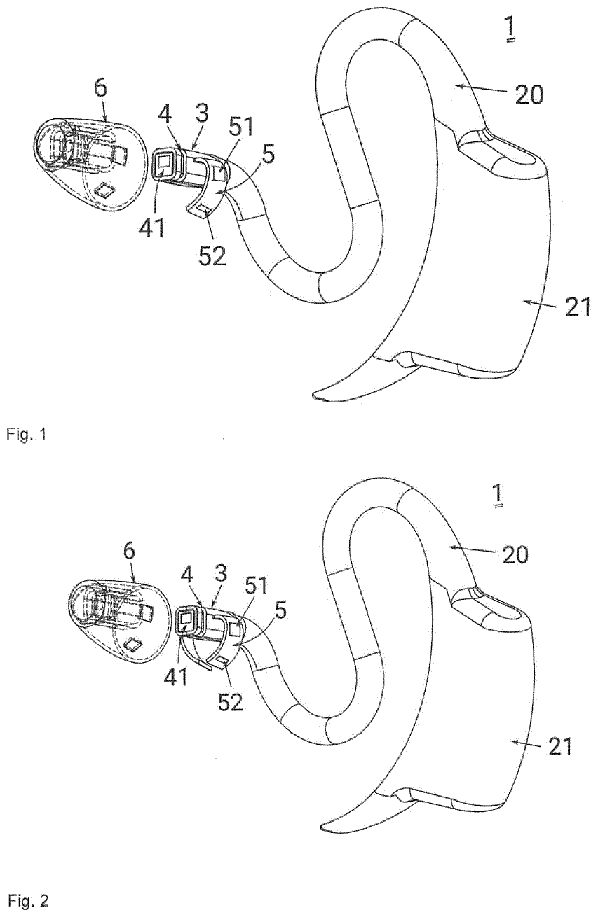

FIG. 1 shows a device 1 for measuring physiological parameters, which can be inserted into an external auditory canal of a human (not shown). The device includes an S-shaped and anatomically adapted hose line 20 for the partial insertion into the external auditory canal of a human and a housing 21 connected to the hose line 20. The housing is designed in such a way that a sensor device for measuring a physiological parameter is integrated into the housing. The hose line 20 includes a section 4 at a distal end 3 that faces the eardrum when worn, the section 4 having a wing element 5 with a first sensor component 51 and a second sensor component 52 for measuring physiological parameters. In this example, the first sensor component is 51 a photodiode and the second sensor component 52 an LED. The wing element 5 is made of an elastomer and has a restoring force due to its intrinsic elastic material properties, so that, when the device is worn, the first sensor component 51 is brought into contact with an inner wall of the external auditory canal by the restoring force of the wing element 5. In this exemplary implementation, the section 4 includes a third sensor component 41, in this case an IR sensor or a loudspeaker, at its distal end 3. When the device 1 is worn, the third sensor component 41 is positioned stably in the external auditory canal. The third sensor component 41 does not touch the inner wall of the external auditory canal when worn, but can carry out the measurements without contact. In the example, there are further sensor components behind the third sensor component 41 and within section 4, in this case an acceleration sensor and a gyroscope for determination of acceleration and rotational movements. The device 1 additionally includes an elastic umbrella 6, in this case an elastic umbrella made of an elastic polymer elastic, for positioning in the external auditory canal, the elastic umbrella 6 being releasably connected to the section 4 via a plug connection.

FIG. 2 shows a device 1 in an alternative design with two similar wing elements 5. In this example, the two wing elements 5 are arranged in a mirror symmetry around the essentially cylindrical section 4. The restoring force acts on the elastic umbrella from two sides, so that the sensor system can be pressed evenly against the inner wall of the hearing. Wearing the device is particularly comfortable in this way.

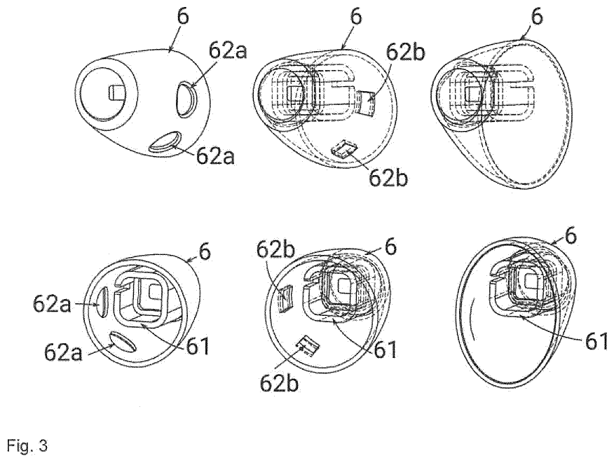

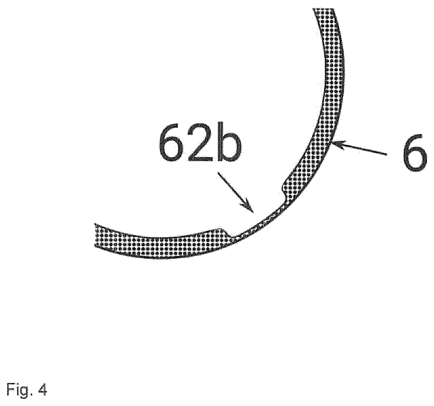

FIG. 3 shows various exemplary implementations of the elastic umbrellas 6. The elastic umbrella 6 has a recess 61 which is adapted in size and shape to accommodate the section 4 therein, whereby the elastic umbrella 6 can be connected to the section 4 in a detachable manner via a plug connection. In a first variant (variant A), the elastic umbrella 6 can have two holes 62a (or side holes) so that, when the device is worn, a first and a second sensor component 51, 52 can be brought into contact with an inner wall of the external auditory canal through the holes 62a. In a second variant (variant B), the elastic umbrella 6 can have two depressions 62b with a residual material with a small material thickness of 10 μm to 2 mm, in particular 200 μm, so that, when the device is worn, a first and a second sensor component 51, 52 can be brought into measuring connection to an inner wall of the external auditory canal via the residual material of the one or more depressions 62b. The residual material of the two depressions 62b is translucent and has a material thickness of the residual material of 10 μm to 2 mm, the first and second sensor components 51, 52 each being a light sensor and/or an LED. In a third variant (variant C), the elastic umbrella has no holes/bores or depressions on the surface. In all three variants, the elastic umbrella is made in one piece and is made from either a clear, colorless or matt, Elastomer interspersed with a few color pigments. The residual material, which is still present through the depression, is also made of the same material in the third variant, but it is made so thin that it is essentially transparent to light, so that contact-free measurements can be carried out by the first and second sensor components in the wearer's external ear canal.

FIG. 4 shows an exemplary implementation of the elastic umbrella 6 of the device with a clear, colorless depression 62b made in a small material thickness of 10 μm or 50 μm. The residual material of the depression 62b is in one piece with the elastic umbrella 6 and is made of the same material. In this example, the elastic umbrella and the residual material of the depression 62b consist of a clear, colorless elastomer.

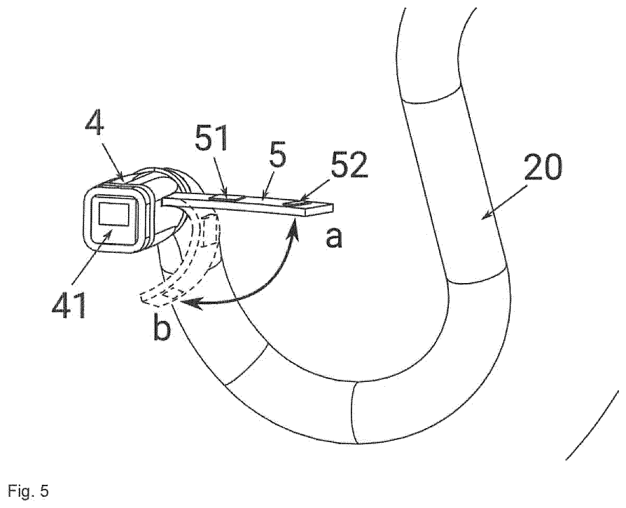

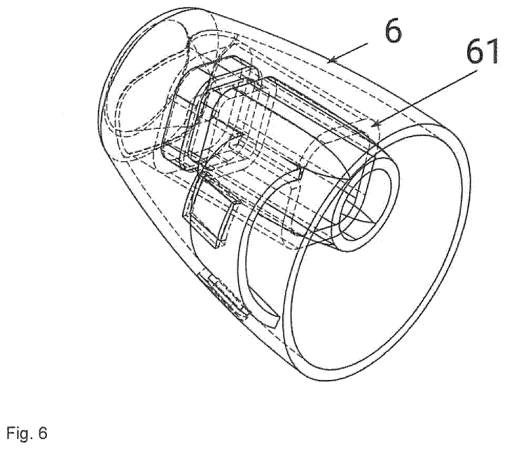

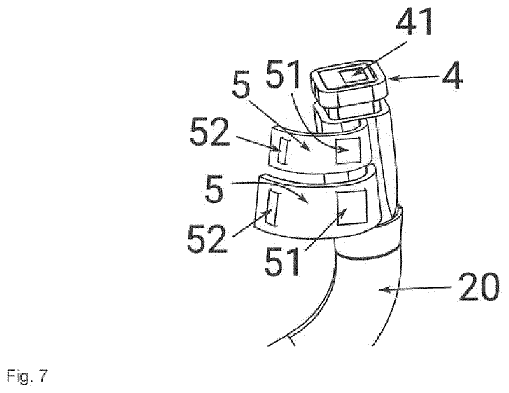

FIG. 5 shows an exemplary implementation of a device for measuring physiological parameters with a wing element and three sensor components. In this exemplary implementation, the wing element 5 has a first sensor component 51 and a second sensor component 52. The first and second sensor components 51, 52 are each a photodiode and/or an LED. When the device is worn, the first and second sensor components 51, 52 are brought into contact with an inner wall or the skin of the external auditory canal by the restoring force of the wing element 5. The at least one wing element 5 consists entirely or essentially of an elastic material, for example an elastomer, which provides an elastic restoring force due to its intrinsic material properties. The spring action of the wing element 5 necessary for pressing the sensor components 51 in the user's external auditory canal can be achieved in that the wing element or elements is/are not bent in the basic position, while they are at least partially bent after positioning in the external auditory canal. The illustration shows the basic position (straight, a) and the curved position (bent, b) of the wing element 5 that will later be positioned in the external auditory canal.

FIG. 6 shows an exemplary implementation of the elastic umbrella 6 of the device 1 with a recess 61 for receiving the section 4. The umbrella can easily be connected to the section 4 by plugging it on, whereby it is held in the section 4 by adhesive and frictional forces. Alternatively, the umbrella 6 can also be connected to section 4 with a screw connection, i.e. in this case the umbrella is screwed onto section 4. The elastic umbrellas 6 can be manufactured and provided in different shapes and sizes, so that even using strict hygiene regulations in pandemic times when using the device with multiple users, the device can be easily changed from one user to another user and can be positioned stably in the external auditory canals of different users. After each use, the elastic umbrella 6 can be separated from section 4 and disposed of as medical waste or in household waste.

If the umbrella 6 is attached from the front, then the wing element 5 can be bent, but this will cause a restoring force to the outside (position b). In the case of an umbrella, the restoring force acts through the umbrella due to the elasticity of the umbrella and presses the umbrella against the skin of the external auditory canal. This ensures direct or indirect contact of the wing element 5 with the external auditory canal. The elastic umbrella 6 and the wing element 5 adapt to the different shapes of the external auditory canal.

FIG. 7 shows an exemplary implementation of a device 1 for measuring physiological parameters with two wing elements 5 arranged one behind the other and two pairs of sensor components 51, 52. In this exemplary implementation, both wing elements 5 have a first sensor component 51 and a second sensor component 52. The first and second sensor components 51, 52 of both wing elements 5 are each a photodiode and an LED. When wearing the device, the first and second sensor components 51, 52 can be brought into contact with an inner wall of the external auditory canal by the restoring force of the wing elements 5. Both wing elements 5 each consist completely or are essentially made of an elastic material, for example an elastomer, which provides an elastic restoring force due to its intrinsic material properties. In the case of the wing elements, the respective restoring forces act in the same direction, so that the wing elements are only pressed against one side of the external auditory canal, while the other side has neither restoring forces nor sensors. In this case, however, the positioning is very stable in the external auditory canal,

FIG. 8 shows an exemplary implementation of a device 1 for measuring physiological parameters with three wing elements 5 arranged next to one another and three pairs of sensor components 51, 52. In this exemplary implementation, all three wing elements 5 each have a first sensor component 51 and a second sensor component 52. The first and second sensor components 51, 52 of all wing elements 5 are each a photodiode and an LED, respectively. All three wing elements 5 each consist completely or are essentially made of an elastic material, for example an elastomer, which provides an elastic restoring force due to its intrinsic material properties.

FIGS. 9 and 10 show two exemplary implementations of a device 1 for measuring physiological parameters with one or two wing elements 5 and several sensor components. FIG. 9 shows a detailed view of an exemplary implementation of a device in which a wing element 5 with a first sensor component 51 and a second sensor component 52 is attached to section 4. The first and second sensor components are arranged next to one another in the distal direction and spatially separated from one another. In this example, the first sensor component 51 is an LED and the second sensor component 52 is a photodiode. The light emitted by the first sensor component 51 cannot reach the second sensor component 52 directly due to the spatial separation. The second sensor component 52 measures physiological parameters such as pulse rate, arterial oxygen saturation, respiratory rate with high resolution directly on the ear contact surface of the external auditory canal.

FIG. 10 shows an exemplary implementation of a device 1 for measuring physiological parameters with two wing elements 5 arranged one behind the other and first and second sensor components 51, 52. In this exemplary implementation, both wing elements 5 have a first sensor component 51 and a second sensor component 52. The first sensor component 51 of both wing elements 5 are each an LED. The second sensor component 52 of both wing elements 5 are each a light sensor, e.g. a photodiode. When wearing the device, the first and second sensor components 51, 52 are brought into contact with an inner wall of the external auditory canal by the restoring force of the wing elements 5. Both wing elements 5 each consist completely or are essentially made of an elastic material, for example an elastomer, which provides an elastic restoring force due to its intrinsic material properties. Thanks to the two wing elements arranged one behind the other, the device can be held in the external auditory canal in a very stable and comfortable manner for the user.

Measuring physiological parameters in a human ear canal can be achieved using the following method. In a first step (step a)), a device 1 for measuring physiological parameters, which can be inserted into an external auditory canal of a human, is provided; in a second step (step b)), the wing element 5 or the wing elements 5 of the section 4 moves and prestresses against his/her restoring force (position b) of FIG. 5). The section 4 is detachably connected to the elastic umbrella 6 by inserting the section 4 into the recess 61 of the elastic umbrella. The at least one wing element 5 remains in its prestressed position, but develops a force on the elastic umbrella and can deform it. The first sensor component 51 and second sensor component 52 come to rest, for example, in the area of the depressions 62b on the residual material of the depressions or, if the elastic umbrella has holes, the first sensor component 51 and second sensor component 52 can be positioned in the holes so that measurements of physiological parameters can be achieved by emitting and receiving light through the holes. In a third step (step c)), the device 1 is inserted into the external auditory canal of a human by bringing the first sensor component 51 and second sensor component 52 into contact with an inner wall or skin of the external auditory canal by the restoring force. The sensor components 51, 52 lie directly on the skin of the user's external auditory canal or, in the case of depressions, are positioned in the optical proximity to the skin of the user's external auditory canal. In a fourth step (step d)), physiological parameters in the external auditory canal of a human are measured using the first sensor component 51 and the second sensor component 52. After the measurement, the device can be pulled out of the user's external auditory canal.

After each use of the device, the elastic umbrella 6 is separated from section 4 and can be disposed of with household waste or as medical waste in accordance with hygiene regulations.

### LIST OF REFERENCE SYMBOLS

1 device
20 hose line
21 housing
3 distal end of the hose line
4 section
41 third sensor component (section)
5 wing element
51 first sensor component (wing element)
52 second sensor component (wing element)
6 elastic umbrella
61 recess
62a holes in the elastic umbrella
62b depressions in the elastic umbrella

1-12. (canceled)

13. Device (1) for measuring physiological parameters which is insertable into an external auditory canal of a human, comprising a hose line (20) for insertion into the external auditory canal of a human and a housing (21) connected to the hose line (20), into which at least a sensor device for measuring a physiological parameter is integrated, the hose line (20) having a section (4) at a distal end (3) facing the eardrum when worn, the section (4) having at least one wing element (5) with at least one first sensor component (51), preferably a photodiode and/or an LED, for measuring physiological parameters, wherein the wing element (5) has a restoring force, so that, when the device is worn, the at least one first sensor component (51) can be brought into contact with an inner wall of the external auditory canal by the restoring force of the wing element (5).

14. Device (1) according to claim 13, wherein the wing element (5) comprises at least one second sensor component (52), preferably a photodiode and/or an LED, so that, when the device is worn, the at least one second sensor component (52) can be brought into contact with an inner wall of the external auditory canal by the restoring force the wing element (5).

15. Device (1) according to claim 13, additionally comprising at least one third sensor component (41) at the distal end (3) of the section (4), wherein, when the device is worn, the at least one third sensor component (41) can be stably positioned in the external auditory canal.

16. Device (1) according to claim 13, additionally comprising an elastic umbrella (6) for positioning in the external auditory canal, wherein the elastic umbrella (6) is detachably connected to the section (4).

17. The device (1) according to claim 16, wherein the elastic umbrella (6) comprises a recess (61) adapted in size and shape to receive the section (4) therein, thereby connecting the elastic umbrella (6) with the section (4) in a detachable manner.

18. Device (1) according to claim 16, wherein the elastic umbrella (6) comprises one or more holes (62a), so that, when the device is worn, the at least one first and optionally the at least one second sensor component (51, 52) can be brought into contact with an inner wall of the external auditory canal through the one or more holes (62a).

19. Device (1) according to claim 16, wherein the elastic umbrella (6) has one or more depressions (62b) with a residual material with a low material thickness, so that, when the device is worn, the at least one first and optionally the at least one second sensor component (51, 52) can be brought into measuring connection to an inner wall of the external auditory canal via the residual material of the one or more depressions (62b).

20. Device (1) according to claim 19, wherein the residual material of the one or more depressions (62b) is translucent and the at least one first and optionally the at least one second sensor component (51, 52) is at least one light sensor.

21. Device (1) according to claim 13, comprising at least two wing elements (5).

22. Device (1) according to claim 13, wherein the at least one wing element (5) essentially consists of an elastic material.

23. Device (1) according to claim 13, wherein the at least one wing element (5) essentially consists of an inelastic material and comprises at least one spring element, for example a spiral spring, for providing a restoring force.

24. Method for measuring physiological parameters in an external auditory canal of a human, comprising the following steps:
a) providing a device (1) for measuring physiological parameters, which is insertable into an external auditory canal of a human, comprising a hose line (20) for insertion into the external auditory canal of a human and a housing (21) connected to the hose line (20), into which at least a sensor device for measuring a physiological parameter is integrated, the hose line (20) having a section (4) at a distal end (3) facing the eardrum when worn, the section (4) having at least one wing element (5) with at least one first sensor component (51), preferably a photodiode and/or an LED, for measuring physiological parameters, wherein the wing element (5) has a restoring force, so that, when the device is worn, the at least one first sensor component (51) can be brought into contact with an inner wall of the external auditory canal by the restoring force of the wing element (5);
b) moving the at least one wing element (5) of the section (4) against its restoring force and optionally releasably connecting the section (4) to the elastic umbrella (6);
c) introducing the device (1) into the external auditory canal of a human, bringing the at least one first sensor component (51) into contact with an inner wall of the external auditory canal by the restoring force of the wing element (5); and
d) measuring physiological parameters in the external auditory canal of a human using the at least one first sensor component (51).
